(19) United States
(12) Patent Application Publication
(10) Pub. No.: US 2025/0205287 A1
(43) Pub. Date: Jun. 26, 2025

(54) METHOD FOR ESTABLISHING A MESENCHYMAL STEM CELL SEED BANK BY MIXING TISSUES FROM DIFFERENT DONOR SOURCES

(71) Applicant: Jiangsu Mengpili Biotechnology Co., Ltd, Changzhou City (CN)

(72) Inventors: Zhaoyang LU, Changzhou City (CN); Jian Shen, Changzhou City (CN); Ziyang GE, Changzhou City (CN); Yan Zhong, Changzhou City (CN); Jie Zhao, Changzhou City (CN)

(21) Appl. No.: 18/984,687

(22) Filed: Dec. 17, 2024

(30) Foreign Application Priority Data
Dec. 22, 2023 (CN) 202311792192.X

(51) Int. Cl. A61K 35/28; C12N 5/00; C12N 5/0775; C12N 9/76

(52) U.S. Cl. CPC A61K 35/28; C12N 5/0037; C12N 5/0662; C12N 5/525; C12N 5/562; C12N 9/6427; C12N 2500/24; C12N 2500/33; C12N 2509/00; C12Y 304/21004

(57) ABSTRACT

A method for establishing a seed cell bank of mesenchymal stem cells (MSC), which belongs to the technical field of stem cell culture. The method includes: mixing a plurality of P2 generation (Pn−1 generation) MSCs from different donor sources for culturing.

### CROSS-REFERENCE TO RELATED APPLICATIONS

This application claims the priority and benefit of Chinese patent application No. 202311792192.X, filed on Dec. 22, 2023. The entirety of Chinese patent application No. 202311792192.X is hereby incorporated by reference herein and made a part of this specification.

### REFERENCE TO AN ELECTRONIC SEQUENCE LISTING

The contents of the electronic sequence listing (SequenceListing.xml; Size: 27,507 bytes; and Date of Creation: Mar. 5, 2025) is herein incorporated by reference.

### TECHNICAL FIELD

The disclosure is related to the technical field of stem cell culture, and in particular to a method for establishing a mesenchymal stem cell seed bank by mixing tissues from different donor sources.

### BACKGROUND ART

Stem cells are primitive cells with the potential for self-replication and multidirectional differentiation. Stem cell technology is an important research field in life sciences in recent years. According to the development stage, stem cells can be classified into embryonic stem cells and adult stem cells. Adult stem cells can be derived from bone marrow, peripheral blood, cord blood, umbilical cord, etc. Among adult stem cells, mesenchymal stem cells (MSC) have been the most widely studied and clinically applied in recent years. Depending on the tissue source from which MSC are isolated, there are bone marrow MSC, adipose MSC, and umbilical cord MSC.

MSCs prepared from a single donor seed cell show huge heterogeneity between donors due to differences in the genetic background of the donors, and there are huge differences between each production batch. The reasons for MSC heterogeneity are not only related to the different genetic backgrounds of donors, but also to the operations during the production process. This heterogeneity manifests itself in many aspects, including cell proliferation, differentiation ability, and immune regulation. In particular, the heterogeneity of MSC growth and proliferation has further affected the advancement of MSC production automation and industrialization. Due to the existence of this heterogeneity, the results of MSC clinical studies are heterogeneous, which increases the difficulty of approval by the competent authorities. Therefore, it can be seen that the huge heterogeneity between MSC production batches is an important technical bottleneck restricting the development of the entire MSC production industry.

Chinese Patent No. 201510454775.0 discloses a method for large-scale production of human umbilical cord mesenchymal stem cells. The method includes the following steps: collecting the umbilical cord and the placenta; separating and extracting hUC-MSC from the umbilical cord, and using the traditional DMEM/F12 culture medium containing fetal bovine serum (FBS) for amplifying culture in the early stage; and using the placental homogenate extraction (PHE) from the same source of the umbilical cord instead of FBS for re-culturing the hUC-MSC in the later stage, so as to obtain the hUC-MSC raw material cells. The production of hUC-MSC can be achieved on a large scale in vitro without using commercial serum-free culture medium. The problem of residual bovine serum is also resolved and a lot of costs is saved. However, the heterogeneity of MSCs has not been resolved.

Chinese Patent 201410325874.4 discloses a method for producing rabbit umbilical cord mesenchymal stem cells, which uses rabbit umbilical cord as raw material, and includes the following steps: cleaning of rabbit umbilical cord tissue, digestion of rabbit umbilical cord tissue, cell inoculation, subculture, immediate use or cryopreservation for standby use. The rabbit umbilical cord mesenchymal stem cells obtained according to the above invention meet the qualified standards for mesenchymal stem cell products through surface antigen detection and multidirectional differentiation identification, and have high proliferation and differentiation potential. The rabbit umbilical cord mesenchymal stem cells obtained according to the above invention provide a new allogeneic transplantation seed cell for stem cell-related application research using rabbits as experimental animals, which greatly ensures the low immunogenicity of umbilical cord mesenchymal stem cells and greatly enriches the existing seed cell bank. However, it is not suitable for clinical use and does not solve the heterogeneity problem of MSCs.

### SUMMARY

In order to solve the problems existing in the existing technology, the disclosure intends to provide a method for establishing a mesenchymal stem cell seed bank, which reduces the heterogeneity of various aspects of MSC performance by optimizing the donor source and the culture environment.

The meaning of “bank” described in the disclosure is: “a collection of qualified cells with the same type and their storage space” or “storing qualified cells with the same type and their space”.

The cells in the “seed cell bank” described in the disclosure refer to Pn generation cells, that is, cells obtained by mixing and culturing the Pn−1 generation. n refers to the total number of passages of mesenchymal stem cells since the primary passage (P0).

The cells in the “raw material cell bank” described in the disclosure refer to unmixed Pn-2 cells from a single donor.

The cells in the “finished cell bank” described in the disclosure refer to Pn+x generation cells, where x≥3, and x refers to the number of passages experienced from the mixed seed cells (Pn generation cells) culture amplifying to the finished cell. The Pn generation cells are obtained by mixed subculture of Pn−1 generation cells from N different donor sources.

In the disclosure, “cell seed bank” and “seed cell bank” have the same concept.

In one aspect, the disclosure provides a method for production a seed cell bank of mesenchymal stem cells.

The production method at least includes: mixing Pn−1 generation mesenchymal stem cells from N different donor sources for culturing to obtain Pn generation mesenchymal stem cells; the N represents the number of donors, N>1; preferably, N>3; further preferably, N>3, N>4, N>5, N>6, N>7, N>8, N>9, N>10, N>11, N>12, N>13, N>14, N>15 or N>16. In some embodiments, N is an integer between 4-16, and may be an integer between 4-9, 9-16, 4-8, 4-7, 5-9, 5-12, 12-16, 8-16, 8-10, 10-12, or 15-16.

Preferably, the mesenchymal stem cells (Pn−1 generation) are the mesenchymal stem cells with a passage ability; they can be mesenchymal stem cells that have been passaged once or more from primary cells, i.e., they can be mesenchymal stem cells that have been passaged for 1 time, 2 times, 3 times, . . . , n−1 times from the primary cells.

Further preferably, the mesenchymal stem cells still have the passage ability for at least more than 4 generations after mixing (Pn generation), preferably more than 6 generations.

Further preferably, the mesenchymal stem cells (Pn generation) are obtained by mixing and culturing Pn−1 generation mesenchymal stem cells that have been passaged 2 or more times from the primary cells, i.e., they can be mesenchymal stem cells that have been passaged 2, 3, 4, . . . , n−1 times from the primary cells. Generally, n≥3.

In some embodiments, the mesenchymal stem cells (Pn−1 generation) are P2 generation cells obtained by passaged twice from the primary cells.

Preferably, the production method includes mixing and culturing a plurality of Pn−1 generation MSCs from different donor sources to obtain Pn generation cells, wherein n represents the total number of passages of the MSC in the seed cell bank, n≥3.

In some embodiments, mixing Pn−1 generation MSCs from N (N=4-16) different donor sources for culture.

Preferably, the Pn−1 generation MSCs from different donor sources are mixed in equal proportions.

The seed cell bank includes Pn generation cells cultured by the aforementioned culture method.

In some embodiments, the mixed MSCs are cultured to generation Pn+1. In actual production, the mixed MSCs can also be cultured to Pn+2, Pn+3, Pn+x . . . generations, as long as it meets the needs of clinical application.

Preferably, the subculture method of the mixed MSCs is: amplifying with a culture medium of MEM-α+5% serum substitute. Further preferably, the culture medium may further include 1-5 mM L-glutamine, and further preferably 2 mM L-glutamine.

In some embodiments, the culture medium further includes 10-20 μM ferric citrate, 6-10 g/L taurine, preferably 10-12 μM ferric citrate, 8-10 g/L taurine.

Preferably, a cell seeding density of the subculture cells is 5E3-1E4/cm2, preferably 5E3/cm2 or 1E4/cm2, further preferably 5E3/cm2.

Alternatively, the Pn−2 generation MSC (raw material cells), are qualified according to following standards:
cell phenotype detection: CD14, CD19, CD34, CD45, MHC-II: ≥98% negative; CD73, CD90, CD105: ≥95% positive; CD3: 100% negative;
differentiation test of chondrocyte, osteoblast and adipocyte: positive;
fungal, bacterial and mycoplasma culture: negative.

A cell viability of the Pn−2 generation MSCs after thawing a cryopreservation tube is ≥80%, preferably ≥85%.

Preferably, the Pn−1 generation MSCs are obtained by amplifying the Pn−2 generation MSCs with a culture medium of MEM-α+5% serum substitute; and the Pn−2 generation MSCs are obtained by digesting the Pn−3 generation MSCs with 0.25% trypsin.

Preferably, the Pn−3 generation MSCs are P0 generation MSCs, and the P0 generation MSCs are obtained by culturing with a culture medium of MEM-α serum-free medium with 5% serum substitute, preferably a culture medium of MEM-α+5% serum substitute+2 mM L-glutamine.

In some embodiments, the production method includes the following steps:
(1) production of P0, P1, . . . , Pn−1 generation MSCs: culturing umbilical cord tissues from different donor sources respectively, washing broken tissue blocks away with physiological saline, and then digesting adhered MSCs with 0.25% trypsin to obtain P0 generation MSCs when MSCs grow from the periphery of the broken tissue blocks of Wharton's Jelly and occupies 60% of a total culture area; continuing the culture in the expanded bottle, and then digesting with trypsin to obtain P1 generation MSCs when the cells grew to occupy 80% of the total culture area; amplifying the P1 generation MSCs with a culture medium of MEM-α+5% serum substitute, digesting with trypsin to obtain P2 generation MSCs; until Pn−2 generation is obtained; cryopreserving the Pn−2 generation (raw material cells) with 10% DMSO as a cryopreserving solution; storing the qualified raw material cells in liquid nitrogen for later use;
(2) thawing Pn−2 generation MSCs (raw material cells), amplifying the Pn−2 generation MSCs with a culture medium of MEM-α+5% serum substitute, and the Pn−1 generation is obtained after trypsin digestion;
(3) Mixing N Pn−1 generation MSCs in raw material cell bank, further culturing and amplifying to obtain Pn generation MSCs, cryopreserving the Pn generation (seed cells) with 10% DMSO as a cryopreserving solution, storing the qualified seed cells in liquid nitrogen for later use.

In another aspect, the disclosure provides a seed cell or a seed cell bank of mesenchymal stem cells.

The seed cells or the seed cell bank of mesenchymal stem cells are obtained by the aforementioned production method.

In another aspect, the disclosure provides a use of aforementioned seed cells or seed cell bank of mesenchymal stem cells as stem cell drugs.

The disclosure also provides a stem cell drug including a finished cell or a finished cell bank obtained by subculturing of aforementioned seed cell or seed cell bank.

The stem cell drug also includes other pharmaceutically acceptable carriers or excipients.

The stem cell drug used for treatment including but not limited to: cerebral palsy in children, multiple sclerosis, graft-versus-host disease in children, Crohn's disease, hematopoietic dysfunction, bone or cartilage damage, nerve damage, muscular dystrophy, diabetes and its complications, osteoarthritis, rheumatoid arthritis, systemic lupus erythematosus, eczema, various liver cirrhosis liver failure, renal failure, acute myocardial infarction, heart failure, cerebral infarction, tumors, retinal macular degeneration, Parkinson's disease, Alzheimer's disease, amyotrophic lateral sclerosis, systemic sclerosis, primary Sjögren's syndrome or dermatomyositis.

In principle, the stem cell drug can be used to treat various types of diseases that can be treated by mesenchymal stem cells disclosed in the existing technology.

The problem to be solved by the disclosure is that the differences in the characteristics of MSCs produced from cells from each single donor source, including cell growth characteristics, should be random and normally distributed. Taking the cumulative cell number of MSCs cultured for 14-28 days as an example, the average value of MSCs produced from each individual donor is A, the standard deviation is S, and the coefficient of variation is CV=S/A. If N donors are mixed to establish a seed bank, and then use the mixed seed cells to produce MSCs; when a plurality (N) of different mixed seed bank cells are used to produce MSCs a plurality of times, a mean value of μ, a standard error of σ, and a coefficient of variation of cv=σ/μ can be obtained based on the obtained values. According to statistical principles, when the times of observations is large enough, μ≈A; σ=S/√N, cv/CV=1/√N. If any 4 donors (N=4) are mixed, the degree of variation of MSCs produced from the mixed seed cells will be half (½) of that produced from a single donor, while if any 9 donors (N=9) are mixed, the degree of variation of MSCs produced from the mixed seed cells will be ⅓ of that produced from a single donor.

Generally speaking, the main obstacle to mixing cells from different individuals with different genetic backgrounds is their immune compatibility, especially the MHC compatibility between immune cells. If immune cells from different donors remain after mixing, a mixed lymphocyte reaction (MLR) will occur between them, and the cytokines produced by this reaction will greatly affect the proliferation and differentiation of MSCs. Fortunately, in the process of producing the raw material cells of the disclosure, lymphocytes have been completely removed, and the storage standard of the raw material cells of the disclosure stipulates that CD3 cells must be 100% negative. In addition, MSCs themselves are a type of low immunogenic cells that do not express MHC-II antigens. In addition, MSCs, especially those derived from the umbilical cord, also have immunosuppressive effects. To sum up, it enable establishing an MSC seed bank by mixing MSC raw material cells from a plurality of donors.

The beneficial effect of the disclosure are in that:
in the disclosure, the mesenchymal stem cell seed bank established by mixing mesenchymal stem cells from different donors can reduce and weaken the differences in MSC growth and proliferation caused by different genetic backgrounds of donors. By stabilizing the results of MSC growth and amplifying, reducing differences between batches, and improving the homogeneity of mesenchymal stem cell products, the necessary conditions are created for the application of large-scale, automated production processes. At the same time, production costs can also be reduced, the promotion of mesenchymal stem cell clinical applications and the development of the entire stem cell therapy industry are promoted.

### BRIEF DESCRIPTION OF THE DRAWINGS

FIG. 1 is a technical route for constructing a mesenchymal stem cell seed bank of the disclosure (construction of a mixed cell seed bank from a plurality of donors and an introduction to the MSC production process).

FIG. 2 is a MSC growth diagram of a single umbilical cord cultured separately for 14 days in Example 1, wherein the numbers represent different umbilical cords.

FIG. 3 is a MSC growth diagram of mixed 4 portions of umbilical cord cultured for 14 days in Example 1, wherein each letter represents a growth diagram of mixed MSC cultures in parallel experiments.

FIG. 4 is a MSC growth diagram of a single umbilical cord cultured separately for 28 days in Example 3, wherein the numbers represent different umbilical cords.

FIG. 5 is a MSC growth diagram of mixed 9 portions of umbilical cord cultured for 28 days in Example 3, wherein each letter represents a growth diagram of mixed MSC cultures in parallel experiments (A mixed −9, representing the Ath experimental result of 9 donor umbilical cord mixed MSC).

FIG. 6 is a photograph of MSC morphology obtained by culturing and amplifying of mixed 9 donor seed cells in Example 3.

FIG. 7 is the results of MSC membrane surface markers (CD73, CD90 and CD105) obtained by culturing and amplifying of mixed 9 donor seed cells in Example 3.

FIG. 8 is the results of MSC membrane surface markers (CD14, CD19, CD34, CD45, and HLA-Dr) obtained by culturing and amplifying of mixed 9 donor seed cells in Example 3.

FIG. 9 is a photograph of MSC (P10) obtained by culturing and amplifying of mixed 9 donor seed cells in Example 3 after triple differentiation and identification by specific staining.

### DETAILED DESCRIPTION

Below take umbilical cord tissue as example in conjunction with specific embodiment, the disclosure is further elaborated in detail, and following embodiment is not used to limit the disclosure, is only used to illustrate the disclosure. Unless otherwise specified, the experimental methods used in the following embodiment, for which specific conditions are not specified in the embodiment are generally carried out under conventional conditions. Unless otherwise specified, the materials, reagents, etc. used in the following embodiment are all commercially available.

Example 1 and Example 2 are preliminary experiments of technical solution of the disclosure.

### Example 1

### 1. MSC Cell Culture

(1) production of umbilical cord MSCs P0 generation, P1 generation and P2 (as Pn−1) generation:

According to the relevant ethical regulations for biological sample collection, the pregnant woman signed a consent form for donating her umbilical cord. 45 umbilical cords from normal pregnant women with negative indicators of various infectious diseases delivered by caesarean section are collected and numbered 1-45. After removing the blood vessels, the umbilical cords are cut into pieces and placed in plastic culture bottles and cultured in MEM-α serum-free medium plus 5% serum substitute (UltraGRO™-Advanced) at 37° C. and 5% CO2. When MSCs grow out from the periphery of the broken tissue pieces and occupy 60% of the total culture area, washing the broken tissue pieces away with physiological saline and then digesting the adherent MSCs with trypsin to obtain P0 generation MSCs at last.

Continuing the culture in the expanded bottle, and then digesting with trypsin to obtain P1 generation MSCs when the cells grew to occupy 80% of the total culture area. Amplifying with a culture medium of MEM-α+5% serum substitute (cell seeding density: 1E4/cm2), digesting with trypsin to obtain P2 generation. cryopreserving the P2 generation MSCs (1E6/tube) with 10% DMSO and stored in liquid nitrogen as a raw material cell bank. Standards for the qualified P2 (Pn−1) generation MSCs are:
CD3: 100% negative;
Cell phenotype detection:
CD14, CD19, CD34, CD45, MHC-II: ≤98% positive;
CD73, CD90, CD105: ≥95% positive;
Differentiation test of three series of cells (chondrocyte, osteoblast, adipocyte): positive;
Fungal, bacterial and mycoplasma culture: negative.
The cell viability of the P2 generation MSCs after thawing a cryopreservation tube: ≥85%.

The raw material cell bank is composed of P2 (Pn−1) generation cells.

(2) production of MSC seed bank (P3 generation): randomly selecting cryopreservation tubes of the P2 generation MSCs from different donor umbilical cords, thawing, and mixing the P2 generation MSCs from different donor umbilical cords in equal proportions to form mixed groups of 4, 9 or 16 different portions of umbilical cords. Culturing and amplifying each single and mixed P2 generation MSCs, respectively. The amplifying method is the same as that of P2 generation, and cryopreserving the P3 generation MSCs of each group (single group and mixed group). The seed cell bank composed of P3 (Pn) generation cells, and the storage standards are as follows:
cell phenotype detection: CD14, CD19, CD34, CD45, MHC-II: ≤98% negative;
CD73, CD90, CD105: 295% positive;
fungal, bacterial and mycoplasma culture: negative;
the cell viability after thawing: ≥85%.

(3) thawing the aforementioned cryopreserved P3 MSCs (single group and mixed group), respectively. Thawing method: placing the cryopreservation tube in a 38° C. water bath, and when the cells have thawed to a size of mung bean-sized ice fragments, transferring the thawed cells to a 15 mL centrifuge tube. Adding 10 mL of MEM-α culture medium, mixing and centrifuging (450G, 5 minutes), removing the supernatant and adding 10 mL of MEM-α culture medium again, and centrifuging again. Culturing the obtained cells with a culture medium of MEM-α+5% serum substitute (cell seeding density: 1E4/cm2). Culturing for 14-21 days, using the same method as P2 amplifying.

The final cultured cells are qualified after inspection to constitute a finished cell bank, and the storage standards are as follows:
Cell phenotype detection:
CD14, CD19, CD34, CD45, MHC-II: ≤98% positive;
CD73, CD90, CD105: ≥95% positive;
Differentiation test of three series of cells (chondrocyte, osteoblast, adipocyte): positive;
fungal, bacterial and mycoplasma culture: negative;
the cell viability after thawing: ≥85%;

Fluorescence in situ hybridization karyotype analysis detects chromosomal aberrations in mixed MSCs cultured by umbilical cord from a plurality of donors: negative.

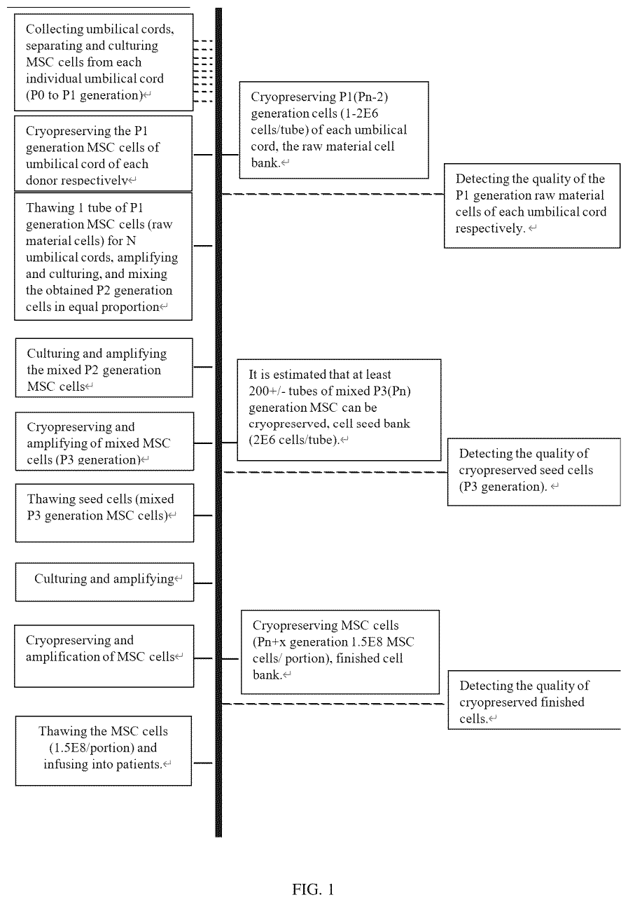

Production route is referring to FIG. 1.

(4) summarizing the results of all single groups into one group (single group), and summarizing the results of all mixed groups together (mixed group-4, mixed group-9, mixed group-16). Comparing the results and the degree of variation (coefficient of variation) between each group.

### 2. Cell Phenotype Detection (Flow Cytometry)

(1) Monoclonal antibodies: CD73-FITC, CD90-PE, CD105-APC, CD14-PC, CD19-PC, CD34-PC, CD45-PC, HLA-Dr-PC and isotype control antibodies are purchased from Biolegend and eBioScience.

(2) MSC cell staining: Prepare 1.5 mL of MSC cell suspension with a concentration of 1E6/mL.

After adding the samples and mixing, placing the 7 tubes aforementioned in a dark place at 4° C. for 30 minutes and washing twice with PBS. Adding 500 μL of PBS to each tube and resuspending the cells.

(3) Flow cytometer (DxFlex, Beckman), after setting the MSC window on the FSC/SSC dot plot, using 1-5 tubes to adjust various parameters including compensation. The 6th tube is used to detect the percentage of three markers CD73, CD90 and CD105 that are simultaneously positive; and the percentage of CD14, CD19, CD34, CD45, and HLA-DR that are negative. The 7th tube is used to detect the viability of MSCs. If the negative percentage of the 6th tube is found to be lower than 98%, 5 more test tubes are needed. Adding CD14, CD19, CD34, CD45, and HLA-DR monoclonal antibodies to each tube respectively, and then adding 100 μL of the remaining cell suspension to each tube. Re-labeling to determine which surface marker has a negative rate below 98%.

(4) P0 generation MSC cells are labeled with CD3-FITC (Biolegend company) monochromatic technology. Taking 2 test tubes, adding CD3-FITC monoclonal antibody and isotype control respectively, and then adding 100 μL of cell suspension (1E6/mL) to each tube. After mixing, placing in a dark place at 4° C. for 30 minutes and washing twice with PBS. Adding 500 μL of PBS to each tube and resuspending the cells. Analyzing the positive ratio of CD3 with flow cytometer (DxFlex, Beckman).

### 3. Results of Example 1:

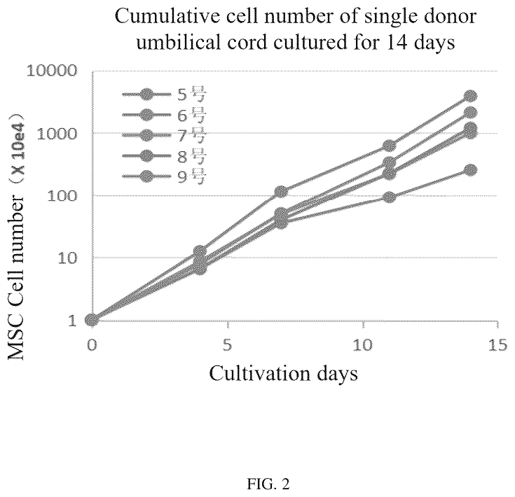

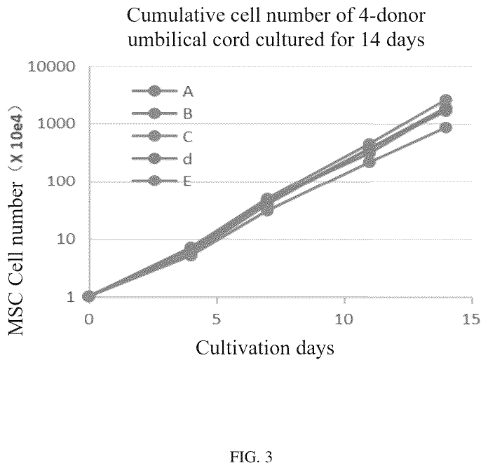

(1) Cell growth and proliferation results are shown in FIG. 2 and FIG. 3.

The amplifying effect statistics are as follows:

| | Number | Average | | Coeffi- |
|---|---|---|---|---|
| | of | ampli- | | cient of |
| | experi- | fying | Standard | variation/ |
| | ments | multiples | deviation | % |
| Traditional culture process | 5 | 1,676 | 1,385 | 82.6 |
| (single umbilical cord | | | | |
| culture) | | | | |
| The disclosure - mixed 4 | 5 | 1,731 | 613 | 35.4 |
| umbilical cords | | | | |
| The disclosure - mixed 9 | 5 | 1,884 | 859 | 45.6 |
| umbilical cords | | | | |
| The disclosure - mixed 16 | 5 | 1,767 | 911 | 51.5 |

The results verified that mixed culturing of umbilical cords from a plurality of donors can reduce and weaken the differences in MSC growth and proliferation caused by different genetic backgrounds of donor umbilical cords, but as the genetic background conditions of the umbilical cord become more complicated, its effect on the average amplifying multiples gradually decreases.

### Example 2

Based on Example 1, in order to make the final cell bank more suitable for clinical use, the disclosure optimizes the MEM-α serum-free medium in step (1) of MSC cell culture. In addition to the original 5% serum substitute (UltraGRO™-Advanced), 10-20 μM ferric citrate and 6-10 g/L taurine are additionally supplemented in the MSC cell culture. Performing MSC culture experiments on umbilical cords from different donor sources, and setting a single additive control group (i.e. adding only ferric citrate or taurine). The specific groups are as follows:

| Group | Additional components added to the culture medium |
|---|---|
| 4-1 | 10 μM ferric citrate + 10 g/L taurine |
| 4-2 | 12 μM ferric citrate + 8 g/L taurine |
| 4-3 | 12 μM ferric citrate |
| 4-4 | 8 g/L taurine |

Using Example 1 as a control, testing the MSCs produced by mixing 4 or 9 umbilical cords from different sources.

The coefficient of variation of multiples of culture amplifying in vitro of mixed 4 umbilical cords (P7) is as follows:

| | Number of | Coefficient of |
|---|---|---|
| | experiments | variation/% |
| Example 1 | 3 | 44.1 |
| 4-1 | 3 | 32.4 |
| 4-2 | 3 | 31.8 |
| 4-3 | 3 | 41.0 |
| 4-4 | 3 | 42.7 |

### Example 3

The method further optimized on the basis of preliminary experiment of Example 1 is as follows:

### 1. MSC Cell Culture

(1) production of umbilical cord MSCs P0 generation, P1 generation and P2 (as Pn−1) generation:

According to the relevant ethical regulations for biological sample collection, the pregnant woman signed a consent form for donating her umbilical cord. 45 umbilical cords from normal pregnant women with negative indicators of various infectious diseases delivered by caesarean section are collected and numbered 1-45. After removing the blood vessels, cutting the umbilical cords into pieces and placing in plastic culture bottles respectively, and culturing in MEM-α serum-free medium supplemented with 5% serum substitute (UltraGRO™-Advanced)+2 mM L-glutamine at 37° C. and 5% CO2, washing broken tissue blocks away with physiological saline, and then digesting adhered MSCs with 0.25% trypsin to finally obtain P0 generation MSCs when MSCs grow from the periphery of the broken tissue blocks and occupies 60% of a total culture area.

Continuing the culture in the expanded bottle, and then digesting with trypsin to obtain P1 generation MSCs when the cells grew to occupy 80% of the total culture area. cryopreserving the P1 generation MSCs (1E6/tube) with 10% DMSO as a cryopreserving solution and stored in liquid nitrogen as raw material cells for storage management. Standards for the qualified P1 (Pn−2) generation MSCs are:
CD3: 100% negative;
Cell phenotype detection:
CD14, CD19, CD34, CD45, MHC-II: 298% negative;
CD73, CD90, CD105: 295% positive;
Differentiation test of three series of cells (chondrocyte, osteoblast, adipocyte): positive;
fungal, bacterial and mycoplasma culture: negative;
The cell viability of the P1 generation MSCs after thawing a cryopreservation tube: ≥80%.

The raw material cell bank is composed of P1 (Pn−2) generation cells.

(2) production of MSC seed bank (P3 generation): randomly selecting 9 P1 generation MSC cryopreservation tubes of umbilical cord from different donors from the raw material cell bank, thawing and amplifying to obtain P2 generation, and mixing the P2 generation MSCs of umbilical cord from the 9 different donors in equal proportions after trypsin digestion.

Culturing and amplifying the corresponding individual and mixed P2 generation MSCs with a culture medium of MEM-α+5% serum substitute+2 mM L-glutamine, digesting with trypsin to obtain P3 generation. Cryopreserving the P3 generation MSCs of each group (single group and mixed group). The seed cell bank is composed of P3 (Pn) generation cells, the storage standard are as follows: cell phenotype detection: CD14, CD19, CD34, CD45, MHC-II: ≥98% negative;
CD73, CD90, CD105: 295% positive;
fungal, bacterial and mycoplasma culture: negative.
the cell viability after thawing: ≥80%;

(3) thawing the aforementioned cryopreserved P3 MSCs (single group and mixed group), respectively. Thawing method: placing the cryopreservation tube in a 38° C. water bath, and when the cells have thawed to a size of mung bean-sized ice fragments, transferring the thawed cells to a 15 mL centrifuge tube. Adding 10 mL of MEM-α culture medium, mixing and centrifuging (450G, 5 minutes), removing the supernatant and adding 10 mL of MEM-α culture medium again, and centrifuging again. Culturing and amplifying the obtained cells with a culture medium of MEM-α+5% serum substitute+2 mM L-glutamine. Culturing for 14-28 days, using the same method as P2 amplifying.

The final cultured cells are qualified after inspection to constitute a finished cell bank, and the storage standards are as follows:
Cell phenotype detection:
CD14, CD19, CD34, CD45, MHC-II: 298% negative;
CD73, CD90, CD105: 295% positive;
Differentiation test of three series of cells (chondrocyte, osteoblast, adipocyte): positive;
fungal, bacterial and mycoplasma culture: negative.
the cell viability after thawing: ≥80%;

Production route is referring to FIG. 1.

(4) repeating the experiment of mixing umbilical cord cells from 9 different donors to produce a seed bank 7 times (different combinations). Summarizing the results of all single groups into one group (single group), and summarizing the results of all mixed groups together. Comparing the results of the single group and mixed group and the degree of variation (coefficient of variation).

### 2. Cell Phenotype Detection (Flow Cytometry)

(1) Monoclonal antibodies: CD3-FITC, CD73-FITC, CD90-PE, CD105-APC, CD14-PerCP, CD19-PerCP, CD34-PerCP, CD45-PerCP, HLA-Dr-PerCP and isotype control antibodies are purchased from Biolegend and eBioScience.

(2) MSC cell staining: Prepare 1.5 mL of MSC cell suspension with a concentration of 1E6/mL.

| Tube | Cell | FITC- | PE- | APC- | PerCP- |
|---|---|---|---|---|---|
| number | (μL) | antibody | antibody | antibody | antibody |
| 1 | 100 | Isotype | Isotype | Isotype | Isotype |
| | | antibody | antibody | antibody | antibody |
| 2 | 100 | CD73- | Isotype | Isotype | Isotype |
| | | FITC | antibody | antibody | antibody |
| 3 | 100 | Isotype | CD90-PE | Isotype | Isotype |
| | | antibody | | antibody | antibody |
| 4 | 100 | Isotype | Isotype | CD105- | Isotype |
| | | antibody | antibody | APC | antibody |
| 5 | 100 | Isotype | Isotype | Isotype | CD73-PerCP |
| | | antibody | antibody | antibody | |
| 6 | 100 | CD73- | CD90-PE | CD105- | CD14-PerCP, |
| | | FITC | | APC | CD19-PerCP, |
| | | | | | CD34-PerCP, |
| | | | | | CD45-PerCP, |
| | | | | | HLA-DR-PerCP |
| 7 | 100 | CD73- | CD90-PE | CD105- | 7-AAD |
| | | FITC | | APC | |

After adding the samples and mixing, placing the 9 tubes aforementioned in a dark place at 4° C. for 30 minutes and washing twice with PBS. Adding 500 μL of PBS to each tube and resuspending the cells.

(3) Flow cytometer (DxFlex, Beckman), after setting the MSC window on the FSC/SSC dot plot, using 1-5 tubes to adjust various parameters including compensation. The 6th tube is used to detect the percentage of three markers CD73, CD90 and CD105 that are simultaneously positive; and the percentage of CD14, CD19, CD34, CD45, and HLA-DR that are negative. The 7th tube is used to detect the viability of MSCs. If the negative percentage of the PerCP fluorescence channel in the 6th tube is found to be lower than 98%, 5 more test tubes are needed. Adding CD14, CD19, CD34, CD45, and HLA-DR monoclonal antibodies to each tube respectively, and then adding 100 μL of the remaining cell suspension to each tube. Re-labeling to determine which surface marker has a negative rate below 98%. In order to better observe the expression status of each negative marker, a single marker staining method is used in this example.

(4) P1 and P3 generation MSC cells are labeled with CD3-FITC (Biolegend company) monochromatic technology. Taking 2 test tubes, adding CD3-FITC monoclonal antibody and isotype control respectively, and then adding 100 μL of cell suspension (1E6/mL) to each tube. After mixing, placing in a dark place at 4° C. for 30 minutes and washing twice with PBS. Adding 500 μL of PBS to each tube and resuspending the cells. Analyzing the positive ratio of CD3 with flow cytometer (DxFlex, Beckman).

### 3. Differentiation Ability of Three Series of Cells (Osteoblast, Chondrocyte, Adipocyte).

Using the Test Kit of Haoyanghua Biotechnology Co., Ltd. (Tianjin).

(1) Osteoblast: performing according to the operation method recommended by the osteogenic differentiation kit of human umbilical cord mesenchymal stem cells (Cat. No. TBD20190002). Seeding the MSC cells to be tested at a density of 4E4/cm2 in a 6-well plate treated with gelatin, and adding 2 mL of MSC complete culture medium and culturing in a 37° C., 5% CO2 incubator until the confluence is 60%. Then culturing the cells with MSC osteogenic differentiation medium for 2-4 weeks, with changing the medium every 3 days. After the culture is completed, washing the culture wells twice with DPBS, fixing with 4% neutral paraformaldehyde for 20 minutes, staining with Alizarin red, and observing the red calcium nodule staining points under a low-power microscope.

(2) Chondrocytes: performing according to the operation method recommended by the chondrogenic differentiation kit of human umbilical cord mesenchymal stem cells (Cat. No. TBD20190003). Centrifugation washing the MSC cells to be tested twice with chondrocyte premix, and then adjusting the cell concentration to 5E5/mL with induction complete medium, adding 500 μL of cell suspension to a 15 mL centrifuge tube, centrifuging at 150 g for 5 minutes, and then culturing in a 37° C., 5% CO2 incubator for 24 hours. Gently flicking the centrifuge tube to suspend the cell pellet. Continuing the culture for 3 weeks, during which the entire old culture medium was replaced with fresh complete induction medium (500 μL) every 2 days. After the culture is completed, washing the cell pellet twice with DPBS, staining with Alcian blue and observing under a microscope.

(3) Adipocyte differentiation: performing according to the operation method recommended by the adipogenesis differentiation kit of human umbilical cord mesenchymal stem cells (Cat. No. TBD20190004). Seeding the MSC cells to be tested at a density of 3E4/cm2 in a 6-well plate treated with gelatin, and adding 2 mL of hUMSC complete culture medium and culturing in a 37° C., 5% CO2 incubator until the confluence is 80%. Discarding the original culture supernatant and replacing with induction solution A. After 72 hours, replacing with induction solution B. After 24 hours, replacing with induction solution A again. Repeat this cycle 3-5 times. When obvious lipid droplets appeared in the cells, stopping the induction solution A and replacing the induction solution B every 2 days. The culture is continued until the lipid droplets are large enough and then terminating the culture. After the culture is completed, washing the culture wells twice with DPBS, fixing with 4% neutral paraformaldehyde for 20 minutes, staining with Oil Red O, and observing large areas of red staining spots (fat) under a low-power microscope.

### 4. Expression Analysis of Immune Regulatory Factors and Some Tissue Differentiation Genes.

Extracting mRNA from the sample to be tested, transcribing into cDNA in vitro, and then quantitatively analyzing the corresponding immune regulatory factors and some differentiation indicators at the gene expression level using real-time fluorescence PCR relative quantitative technology.

(1) mRNA extraction: performing according to the recommended operation method of the RNeasy kit of Qiagen (Cat. No. 74104). Collecting 5E6 cells, centrifuging and then adding 350 μL of RLT solution and 350 μL of 70% ethanol. Loading the mixed solution onto the RNeasymini adsorption column, and washing the adsorption column with RW1 and RPE solutions respectively. Finally, passing pure water through the column and collecting the purified mRNA. Measuring the concentration of the collected mRNA with a fluorometer (Qubit 3.0 Fluorometer, Invitrogen company) according to the recommended operating method of the Qubit™ RNA BR Assay Kits from Invitrogen company (Cat. No.: Q10210).

(2) Reverse transcription cDNA production: performing according to the recommended operation method of the PrimeScript™ RT reagent Kit from Takara (Cat. No. RR037A). At first, preparing the RT reaction solution (polythymidine, random primers, mRNA sample, reverse transcriptase and buffer), reaction conditions: 37° C., 15 minutes; 85° C., 5 seconds; 4° C.

(3) Chimeric fluorescence PCR relative quantitative detection method: performing according to the recommended operation method of the dye method color fluorescence quantitative PCR premix of Shanghai Titan Technology Co., Ltd (Cat. No.: G8047-1). The primers used in this project are shown in the table (primer synthesis is commissioned to Shanghai Sangon Biotechnology Co., Ltd.). The reaction volume is 20 μL, running for 35 cycles (95° C., 10 seconds; 60° C., 30 seconds; LineGene 9600 fluorescence quantitative PCR detection system, Hangzhou Bioer Technology Co., Ltd.) after 95° C., 15 minutes of hot start enzyme activation. First, detecting the Ct value of each sample template cDNA with an internal reference gene, and adjusting the concentration of each sample template cDNA according to the obtained Ct value, so that the adjusted Ct value of each template internal reference gene is around 15.

The target gene primer sequences are as follows (Shanghai Sangon Biotechnology Co., Ltd.):

| Type | Gene | Primer (5′ to 3′) | Sequence Number |
|---|---|---|---|
| Reference gene | GAPDH | F: CGG AGT CAA CGG ATT TGG TCG TAT | SEQ ID NO. 1 |
| | | R: AGC CTT CTC CAT GGT GGT | SEQ ID NO. 2 |
| Osteogenic | RUNX2 | F: CCC AGT ATG AGA GTA GGT GTC C | SEQ ID NO. 3 |
| differentiation | | R: GGG TAA GAC TGG TCA TAG GAC C | SEQ ID NO. 4 |
| | BGLAP | F: CGC TAC CTG TAT CAA TGG CTG G | SEQ ID NO. 5 |
| | | R: CTC CTG AAA GCC GAT GTG GTC A | SEQ ID NO. 6 |
| Chondrogenic | BMP2 | F: GCA TCC TCT CCA CAA | SEQ ID NO. 7 |
| differentiation | | R: GCC ACA ATC CAG TCA | SEQ ID NO. 8 |
| | SOX9 | F: AGG AAG CTC GCG GAC CAG TAC | SEQ ID NO. 9 |
| | | R: GGT GGT CCT TCT TGT GCT GCA C | SEQ ID NO. 10 |
| Adipogenic | PPARγ | F: CCA AGA CAT TCC ATT CAC AAG | SEQ ID NO. 11 |
| differentiation | | R:R: CTC CAC AGA CAC GAC ATT C | SEQ ID NO. 12 |
| Stem cell gene | NANOG | F: CTC CAA CAT CCT GAA CCT CAG C | SEQ ID NO. 13 |
| | | R: CGT CAC ACC ATT GCT ATT CTT CG | SEQ ID NO. 14 |
| | SOX2 | F: GCT ACA GCA TGA TGC AGG ACC A | SEQ ID NO. 15 |
| | | R: TCT GCG AGC TGG TCA TGG AGT T | SEQ ID NO. 16 |
| Mitochondrial | ATF4 | F: TTC TCC AGC GAC AAG GCT AAG G | SEQ ID NO. 17 |
| stress-regulated | | R: CTC CAA CAT CCA ATC TGT CCC G | SEQ ID NO. 18 |
| genes | | | |
| cytokine | IL-4 | F: CCG TAA CAG ACA TCT TTG CTG CC | SEQ ID NO. 19 |
| | | R: GAG TGT CCT TCT CAT GGT GGC T | SEQ ID NO. 20 |
| | TGF-β1 | F: CAA GGG CTA CCA TGC CAA CT | SEQ ID NO. 21 |
| | | R: AGG GCC AGG ACC TTG CTG | SEQ ID NO. 22 |
| | HGF | F: CGC TAC GAA GTC TGT GAC ATT CC | SEQ ID NO. 23 |
| | | R: TTC CCC ATT GCA GGT CAT G | SEQ ID NO. 24 |
| enzyme | COX1 | F: GAT GAG CAG CTT TTC CAG ACG AC | SEQ ID NO. 25 |
| | | R: AAC TGG ACA CCG AAC AGC AGC T | SEQ ID NO. 26 |
| | COX2 | F: CGG TGA AAC TCT GGC TAG ACA G | SEQ ID NO. 27 |
| | | R: GCA AAC CGT AGA TGC TCA GGG A | SEQ ID NO. 28 |
| | PTGES2 | F: CTT CCT TTT CCT GGG CTT CG | SEQ ID NO. 29 |
| | | R: GAA GAC CAG GAA GTG CAT CCA | SEQ ID NO. 30 |

The results of fluorescent quantitative PCR are expressed in the form of “ΔCt”, where ΔCt=Ct (target gene)−Ct (reference gene).

5. Fluorescence In Situ Hybridization Karyotype Analysis Detects Chromosomal Aberrations in Mixed MSCs Cultured by Umbilical Cord from a Plurality of Donors. This Project is Commissioned Suzhou Feifan Testing Company to Test MSC Cells.

### Results of Example 3:

1. Cell Growth and Cell Morphology

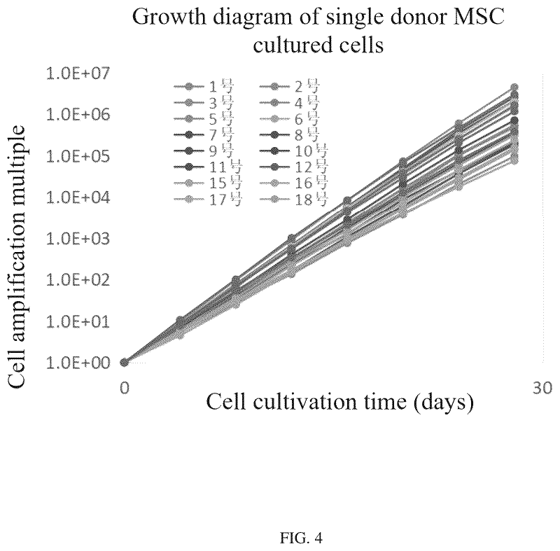

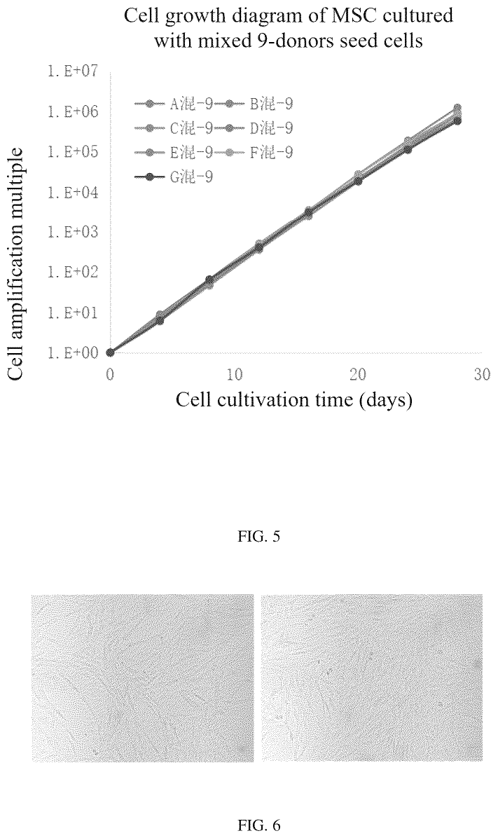

(1) Cell growth and proliferation results are shown in FIG. 4 and FIG. 5.

FIG. 4 and FIG. 5 respectively show the cell growth diagrams of MSCs cultured with seed cells from a single donor and seed cells from mixed 9 donors. In order to verify the stability of the quality of MSCs produced by mixing umbilical cords from donors with different genetic backgrounds, 45 umbilical cords from donors are numbered in this example. When preparing seed cells from mixed donor, randomly selecting and mixing are performed among these 45 donors. In this example, the experiment of mixing umbilical cords from 9 donors to prepare seed cells and then culturing and Amplifying is repeated 7 times in total. In addition, in order to evaluate the differences in MSC culture amplifying caused by different genetic backgrounds of single donor seed cells, the corresponding donor seed cells used in the 7 times of mixed donor umbilical cord seed cell experiment are cultured separately as a control. A total of 22 donor umbilical cords (50% of the total number of umbilical cords) are selected for this example. The results showed that the production of seed cells by mixing a plurality of donors could significantly reduce and weaken the differences in MSC growth and amplifying caused by the different genetic backgrounds of the umbilical cords of each donor. After 16 days of culturing and amplifying (P7), the coefficient of variation of the amplifying multiples decreased from 77.31 for the single-donor seed cell method to 10.78 for the 9-donor mixed seed cell method; and after 28 days of culturing and amplifying (P10), the coefficient of variation of the amplifying multiples decreased from 111.72 to 27.91 (see Table 1).

TABLE 1
Summary and comparison of MSC amplifying results
from single donor and mixed 9 donor seed cells

| Type of seed cell | single donor | | mixed 9 donors | |
|---|---|---|---|---|
| Number of sample | 22 | | 7 | |
| Cell generation | P7 | P10 | P7 | P10 |
| Mean amplification | 3.10E+3 | 1.03E+6 | 3.07E+3 | 7.99E+5 |
| fold | | | | |
| Minimal | 7.44E+2 | 7.43E+4 | 2.50E+3 | 5.63E+5 |
| amplification fold | | | | |
| Maximum | 8.32E+3 | 4.40E+6 | 3.56E+3 | 1.23E+6 |
| amplification fold | | | | |
| Standard deviation | 2.40E+3 | 1.15E+6 | 3.31E+2 | 2.23E+5 |
| coefficient of | 77.31 | 111.72 | 10.78 | 27.91 |
| variation (CV, %) | | | | |

(2) The morphology of MSCs obtained by culturing and amplifying of mixed 9-donor seed cells is shown in FIG. 6.

The obtained MSCs grew in a fibroblast-like adherent manner and had a spindle-shaped or irregular triangle shape with an oval nucleus in the center.

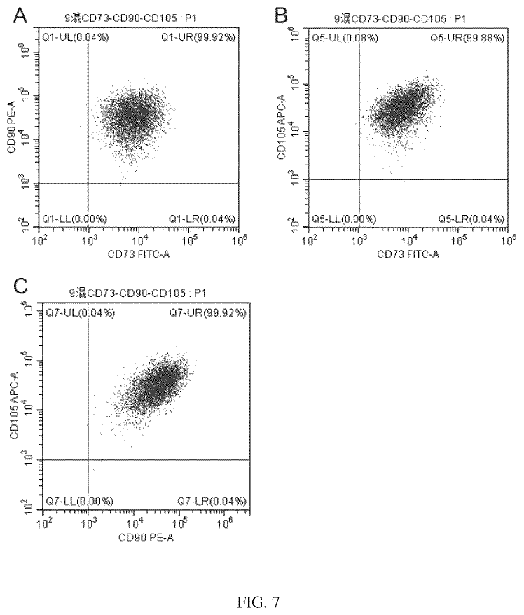

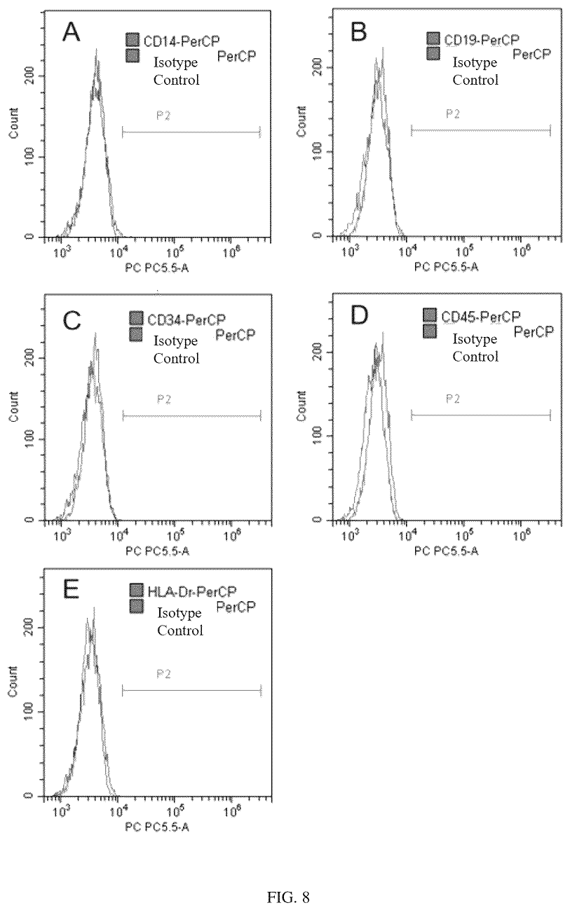

2. Surface markers of MSC (P10) obtained by culturing and amplifying of mixed 9-donor seed cells are shown in FIG. 7 and FIG. 8.

The obtained MSCs simultaneously expressed CD73, CD90 and CD105 on their surface (>95%, see A-C in FIG. 7). Its surface CD14, CD19, CD34, CD45, and HLA-Dr are all negative (see A-E in FIG. 8).

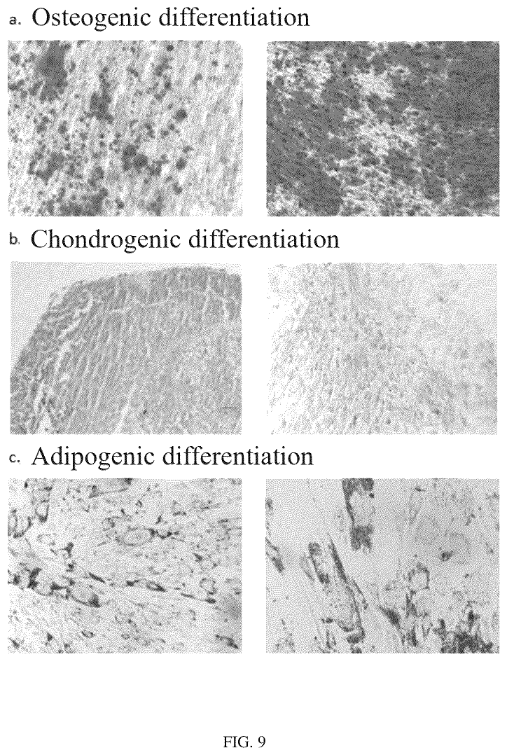

3. The osteogenic, chondrogenic and adipogenic triple differentiation test results of MSC (P10) obtained by culturing and amplifying of mixed 9-donor seed cells are shown in FIG. 9. The results showed that P10 generation MSCs have the potential for multidirectional differentiation, including osteogenic, chondrogenic and adipogenic.

The above results show that the stem cells obtained by mixed donor cell seed culture all meet the standards formulated by the International Association for Cellular Therapy for mesenchymal stem cells (Dominici M. et al. Minimal standard for defining multipotent mesenchymalstromal cells. The International Society for Cellular Therapy position statement. Cytotherapy (2006) 8: (4), 315-317) in terms of cell morphology, cell surface marker and cell differentiation potential.

4. PCR detection of partial differentiation marker genes and immune regulatory factor analysis (see Table 2), the results are expressed in the form of ΔCt. The results showed that the marker genes of osteogenic, chondrogenic and adipogenic differentiation as well as pluripotent stem cells are expressed at low levels in the MSCs cultured from the two groups of different seed cells, and there was no significant difference in the expression level between the two groups (p<0.05). Some genes involved in the regulation of immunity and inflammation (such as TGF-b and COX2) and genes involved in the regulation of embryonic development (such as SOX2) are expressed to a moderate extent in both groups of cultured MSCs. Likewise, there was no significant difference in the expression levels of these genes between the two groups (p<0.05). Therefore, whether culturing with a single donor seed cell or mixed seed cell does not affect the expression of MSC marker genes. However, the degree of difference in the expression of each marker gene in the single donor seed cell culture group (CV1) is greater than that in the mixed donor seed cell culture group (CV2). The degree of difference in the expression of individual marker genes (CV1/CV2) is 3-4 times (for example BMP2: 3.03; SOX2: 4.17). TGF-b1 is a multifunctional cell activity regulator, especially a cytokine that plays a very important role in regulating immune responses. Its overall expression in MSC is higher than that of other tested genes, and the difference in expression between MSCs cultured from single donor seed cells is relatively large, while the difference in expression is weakened in mixed donor seed cells (CV1/CV2=2.97). In conclusion, the marker gene PCR detection confirmed from the gene expression level that the disclosure can effectively reduce the degree of difference in MSC quality by culturing mixed donor seed cells, while the type of seed cells (whether single donor or mixed donor) has no effect on the characteristics of the cultured MSCs itself.

TABLE 2
Cytokine expression of MSCs (P10) amplified by single
donor seed cells and 9-donor mixed seed cells

| | single donor seed cells | | | 9-donor mixed seed cells | | | |
|---|---|---|---|---|---|---|---|
| Target | Average | | CV1 | Average | | CV2 | |
| gene | ΔCt | SD | (%) | ΔCt | SD | (%) | CV1/CV2 |
| Runx2 | 14.39 | 1.21 | 8.40 | 14.32 | 0.53 | 3.68 | 2.28 |
| BGLAP | 14.77 | 1.18 | 8.00 | 15.01 | 0.46 | 3.05 | 2.63 |
| BMP2 | 15.81 | 1.72 | 10.85 | 15.74 | 0.56 | 3.58 | 3.03 |
| Sox9 | 16.30 | 1.65 | 10.14 | 17.20 | 0.97 | 5.63 | 1.80 |
| PPARγ | 14.43 | 1.29 | 8.92 | 13.60 | 0.68 | 4.98 | 1.79 |
| NANOG | 16.73 | 1.54 | 9.23 | 17.23 | 1.47 | 8.53 | 1.08 |
| Sox2 | 14.37 | 2.21 | 15.38 | 15.39 | 0.57 | 3.69 | 4.17 |
| ATF4 | 5.95 | 1.04 | 17.52 | 5.28 | 0.36 | 6.76 | 2.59 |
| TGF-b1 | 8.90 | 0.97 | 10.88 | 8.98 | 0.33 | 3.67 | 2.97 |
| IL-4 | 16.65 | 1.62 | 9.75 | 17.45 | 0.62 | 3.53 | 2.76 |
| HGF | 12.28 | 1.15 | 9.38 | 12.41 | 1.00 | 8.02 | 1.17 |
| PTGES2 | 12.35 | 0.96 | 7.79 | 12.15 | 0.56 | 4.60 | 1.69 |
| COX1 | 13.97 | 0.93 | 6.67 | 13.44 | 0.79 | 5.86 | 1.14 |
| COX2 | 8.48 | 1.08 | 12.79 | 9.43 | 0.90 | 9.52 | 1.34 |

5. MSCs obtained by culturing and amplifying of mixed 9-donor seed cells (P10) are tested for chromosome aberration by a third party (Feifan Standard Technology Service (Suzhou) Co., Ltd.). By G-banding karyotype analysis, analyzing 100 cells, and the karyotype of 72 cells is 46,XY; the karyotype of 18 cells is 46,XX. No abnormalities are found in the chromosome structure.

### Example 4

In order to observe the relationship between the number of mixed donors and the difference in MSC growth and proliferation (coefficient of variation), in addition to mixing 9 donors in Example 3, also performing experiments to culture MSCs by mixing 4- and 16-donor seed cells. The obtained results are consistent with the results of culturing MSCs by mixing 9-donor seed cells (see Table 3).

In order to observe the proportion of each original donor umbilical cord in the mixed group after amplifying, Dishuo Baker Medical Laboratory Co., Ltd. Is particularly commissioned to use the STR-PCR method to perform donor traceability detection on a MSC sample obtained by culturing seed cells mixing four different donors (P10 generation).

The results (see Table 3) further verified that mixed culturing of MSCs from a plurality of donors can reduce and weaken the differences in MSC growth and proliferation caused by different donor genetic backgrounds, and the more donors mixed (N), the effect of reducing differences more obvious, and the effect is inversely proportional to the square root of N. Therefore, theoretically, mixing 9 donors can reduce the differences to 33%. By mixing 4, 9, 16, 25, 36, . . . donors, the differences can be reduced to 50%, 33%, 25%, 20%, 16%, . . . of the original respectively. As N increases, its differential effects on MSC growth and amplifying multiples will gradually decrease. In actual situations, the number of mixed donors (N) should be determined based on the tolerance of the entire production process to differences or the requirements for differences in the final product.

TABLE 3
Summary and comparison of MSC amplifying results
from single donor and mixed 9 donor seed cells

| | Seed cell type | | | |
|---|---|---|---|---|
| | single | 4-mix | 9-mix | 16-mix |
| | donor | donors | donors | donors |
| Number of samples | 22 | 3 | 7 | 3 |
| Cell generation | P10 | | | |
| Mean expansion fold | 1.03E+6 | 8.96E+5 | 7.99E+5 | 1.15E+6 |
| Coefficient of | 111.72 | 60.32 | 27.91 | 21.79 |
| variation (CV, %) | | | | |

In order to detect the growth situation of MSCs derived from each donor seed cell when the mixed donor seed cell culturing and amplifying, a third party (Dishuo Baker Medical Laboratory Co., Ltd.) is commissioned to use the STR-PCR method to detect the proportion of cells from each original donor source after amplifying MSCs with 4-donor mixed seed cell (P10). The results are shown in Table 4. The results showed that each individual umbilical cord still grew and proliferated according to its original characteristics when culturing and amplifying with mixed donor seed cells. Since each individual seed cell has different characteristics, the amplifying multiples weaken or cancel each other out. Therefore, by mixing a plurality of donor seed cells, the goal of improving the uniformity of MSC cell products is achieved overall.

TABLE 4
Proportion of cells from each donor source after mixing
4 kinds of donor seed cells to Amplify MSCs (P10)

| Donor number | Donor-1 | Donor-2 | Donor-3 | Donor-4 |
|---|---|---|---|---|
| Proportion of umbilical cord | 7.58 | 36.03 | 17.74 | 38.65 |
| from a single donor (%) | | | | |

### Example 5

Thawing and amplifying the cryopreserved seed cells in the cell seed bank mixing 9 donors constructed in Example 3 a plurality of times to detect the repeatability of the results obtained using the mixed cell seed bank in the disclosure. In Example 3, a total of 7 cell seed banks mixing 9 donors are constructed. Randomly selecting three of the mixed cell seeds, and thawing, culturing and amplifying five times. The results obtained after 28 days culturing (P10 generation) (see Table 5) showed that the MSC amplifying results obtained using the mixed cell seed bank had good repeatability and stability, and the coefficient of variation of cell amplifying multiples of the three mixed cell seed banks from the same source are all below 10%.

TABLE 5
Repeatability of MSC production results from mixed cell seed bank

| | Repeat | | Standard | Coefficient of |
|---|---|---|---|---|
| | number | Mean | deviation | variation (%) |
| A mixed-9 | 5 | 8.48E+05 | 5.83E+04 | 6.87 |
| C mixed-9 | 5 | 7.71E+05 | 5.97E+04 | 7.75 |
| F mixed-9 | 5 | 7.88E+05 | 5.87E+04 | 7.46 |

### Example 6

On the basis of Example 3 and in combination with the verification results of Example 2, in order to make the final cell bank more suitable for clinical use, the disclosure optimizes the MEM-a serum-free medium in step (1) of MSC cell culture. In addition to the original 5% serum substitute (UltraGRO™-Advanced) and 2 mM L-glutamine, 10-20 μM ferric citrate and 6-10 g/L taurine are additionally supplemented. Performing MSC culture experiments on umbilical cords from different donor sources, and setting a single additive control group (i.e. adding only ferric citrate or taurine). The specific groups are as follows:

TABLE 6
Serum culture medium optimization experiment grouping

| | Additional components added to the |
|---|---|
| Group | culture medium |
| 6-1 | 10 μM ferric citrate + 10 g/L taurine |
| 6-2 | 12 μM ferric citrate + 8 g/L taurine |
| 6-3 | 12 μM ferric citrate |
| 6-4 | 8 g/L taurine |

Using Example 3 as a control, testing the MSCs produced by mixing 4 or 9 umbilical cords from different sources. The coefficient of variation of multiples of culture amplifying in vitro of mixed 4 umbilical cords (P10) is as follows:

TABLE 7
Serum culture medium optimization experiment results

| | Number of | Coefficient of |
|---|---|---|
| | experiments | variation/% |
| Example 3 | 3 | 60.3 |
| 6-1 | 3 | 56.4 |
| 6-2 | 3 | 60.1 |
| 6-3 | 3 | 58.3 |
| 6-4 | 3 | 55.9 |

It should be noted that the above specific implementation are merely some specific forms of implementing the technical solution of the disclosure and are not intended to limit the disclosure. Any technical means substitution or routine adjustment made by those skilled in the art based on the technical solution of the disclosure shall fall within the protection scope of the disclosure.

The above are only preferred embodiments of the disclosure. For those skilled in the art, without departing from the principle of the disclosure, the disclosure can also be improved and modified, and these should also be subject to the protection scope of the disclosure. Mesenchymal stem cells obtained by using the principles of the disclosure are all within the protection scope of the disclosure.

What is claimed is:

1. A method for establishing a seed cell bank of mesenchymal stem cells, comprising: mixing and culturing mesenchymal stem cells from N different donor sources to obtain a seed cell bank, wherein N represents a number of the different donor sources, and N>1.

2. The method according to claim 1, wherein the mesenchymal stem cells are mesenchymal stem cells with a passage ability.

3. The method according to claim 2, wherein the mesenchymal stem cells are mesenchymal stem cells that have been passaged once or more from primary cells.

4. The method according to claim 3, wherein the mesenchymal stem cells have the passage ability for at least more than 4 generations after mixing.

5. The method according to claim 3, wherein the mesenchymal stem cells have the passage ability for at least more than 6 generations after mixing.

6. The method according to claim 4, wherein mixing and culturing comprises mixing and culturing a plurality of Pn−1 generation mesenchymal stem cells (MSCs) from the different donor sources to obtain Pn generation cells, wherein n represents a number of passages of seed cell MSC, and n≥2.

7. The method according to claim 6, wherein the Pn−1 generation MSCs from the different donor sources are mixed in equal proportions.

8. The method according to claim 7, wherein a subculture method of the Pn−1 generation MSCs from the different donor sources after mixing is: amplifying using a culture medium of minimum essential medium α+5% serum substitute (MEM-α+5% serum substitute).

9. The method according to claim 8, wherein the culture medium further comprises 10-20 μM ferric citrate and 6-10 g/L taurine.

10. The method according to claim 9, wherein a cell seeding density of the subculture is 5E3/cm²-1E4/cm².

11. The method according to claim 6, wherein Pn−2 generation MSCs are raw material cells and are qualified according to following standards:
cell phenotype detection: cluster of differentiation (CD) 14 (CD14), CD19, CD34, CD45, MHC-II: ≥98% negative; CD73, CD90, CD105: ≥95% positive; CD3: 100% negative;
differentiation test of chondrocyte, osteoblast or adipocyte: positive;
fungal, bacterial or mycoplasma culture: negative.

12. The method according to claim 11, wherein a cell viability of Pn−2 generation MSCs after thawing a cryopreservation tube is ≥80%.

13. The method according to claim 6, wherein Pn−2 generation MSCs are obtained by amplifying Pn−3 generation MSCs using a culture medium of minimum essential medium α+5% serum substitute (MEM-α+5% serum substitute).

14. The method according to claim 6, wherein Pn−2 generation MSCs are obtained by amplifying Pn−3 generation MSCs using a culture medium of minimum essential medium α+5% serum substitute (MEM-α+5% serum substitute)+2 mM L-glutamine.

15. The method according to claim 13, wherein the Pn−3 generation MSCs are P0 generation MSCs, and the P0 generation MSCs are obtained by culturing with a culture medium of minimum essential medium α+5% serum substitute (MEM-α+5% serum substitute).

16. The method according to claim 13, comprising following steps:
(1) production of P0, P1, . . . , Pn−1 generation MSCs: culturing tissues from the different donor sources respectively, washing broken tissue blocks away with physiological saline, and then digesting adhered MSCs with 0.25% trypsin to obtain P0 generation MSCs when MSCs grow from a periphery of the broken tissue blocks and occupies 60% of a total culture area; continuing the culture in an expanded bottle, and then digesting with trypsin to obtain P1 generation MSCs when the cells grew to occupy 80% of the total culture area; amplifying the P1 generation MSCs with the culture medium of MEM-α+5% serum substitute, digesting with the trypsin to obtain P2 generation MSCs, cryopreserving the Pn−2 generation MSCs and storing in liquid nitrogen as raw material cells with 10% dimethyl sulfoxide (DMSO) as a cryopreserving solution;
(2) detecting the Pn−2 generation MSCs, according to following standards:
cell phenotype detection: cluster of differentiation (CD) 14 (CD14), CD19, CD34, CD45, MHC-II: ≥98% negative; CD73, CD90, CD105: ≥95% positive; CD3: 100% negative;
differentiation test of chondrocyte, osteoblast or adipocyte: positive;
fungal, bacterial or mycoplasma culture: negative;
(3) thawing the qualified Pn−2 generation as raw material cells for culturing and amplifying, and the Pn−1 generation MSCs are obtained after trypsin digestion;
(4) mixing N Pn−1 generation MSCs in equal proportions, then culturing and amplifying to obtain Pn generation MSCs as seed cells, cryopreserving the seed cells with 10% DMSO as a cryopreserving solution, and storing the qualified seed cells in liquid nitrogen;
standards for the qualified Pn generation MSCs as seed cells are:
cell phenotype detection: CD14, CD19, CD34, CD45, MHC-II: ≥98% negative; CD73, CD90, CD105: ≥95% positive.

17. A seed cell or a seed cell bank of mesenchymal stem cells obtained by the method according to claim 1.

18. Use of the seed cells or seed cell bank according to claim 15 as stem cell drugs.

19. A stem cell drug, comprising a finished cell or a finished cell bank obtained by subculturing of the seed cell or seed cell bank according to claim 17;
standards of the qualified finished cells are:
cell phenotype detection: CD14, CD19, CD34, CD45, MHC-II: ≥98% negative; CD73, CD90, CD105: ≥95% positive; differentiation test of chondrocyte, osteoblast or adipocyte:
positive; fungal, bacterial and mycoplasma culture: negative.

20. The stem cell drug according to claim 19, used to treat cerebral palsy in children, multiple sclerosis, graft-versus-host disease in children, Crohn's disease, hematopoietic dysfunction, bone or cartilage damage, nerve damage, muscular dystrophy, diabetes and its complications, osteoarthritis, rheumatoid arthritis, systemic lupus erythematosus, eczema, various liver cirrhosis liver failure, renal failure, acute myocardial infarction, heart failure, cerebral infarction, tumors, retinal macular degeneration, Parkinson's disease, Alzheimer's disease, amyotrophic lateral sclerosis, systemic sclerosis, primary Sjögren's syndrome or dermatomyositis.


SEQUENCE LISTING (30 sequences)

SEQ ID NO: 1 - DNA, 24 bp, synthetic construct
  source 1..24: mol_type = other DNA
cggagtcaacggatttggtcgtat

SEQ ID NO: 2 - DNA, 18 bp, synthetic construct
  source 1..18: mol_type = other DNA
agccttctccatggtggt

SEQ ID NO: 3 - DNA, 22 bp, synthetic construct
  source 1..22: mol_type = other DNA
cccagtatgagagtaggtgtcc

SEQ ID NO: 4 - DNA, 22 bp, synthetic construct
  source 1..22: mol_type = other DNA
gggtaagactggtcataggacc

SEQ ID NO: 5 - DNA, 22 bp, synthetic construct
  source 1..22: mol_type = other DNA
cgctacctgtatcaatggctgg

SEQ ID NO: 6 - DNA, 22 bp, synthetic construct
  source 1..22: mol_type = other DNA
ctcctgaaagccgatgtggtca

SEQ ID NO: 7 - DNA, 15 bp, synthetic construct
  source 1..15: mol_type = other DNA
gcatcctctccacaa

SEQ ID NO: 8 - DNA, 15 bp, synthetic construct
  source 1..15: mol_type = other DNA
gccacaatccagtca

SEQ ID NO: 9 - DNA, 21 bp, synthetic construct
  source 1..21: mol_type = other DNA
aggaagctcgcggaccagtac

SEQ ID NO: 10 - DNA, 22 bp, synthetic construct
  source 1..22: mol_type = other DNA
ggtggtccttcttgtgctgcac

SEQ ID NO: 11 - DNA, 21 bp, synthetic construct
  source 1..21: mol_type = other DNA
ccaagacattccattcacaag

SEQ ID NO: 12 - DNA, 19 bp, synthetic construct
  source 1..19: mol_type = other DNA
ctccacagacacgacattc

SEQ ID NO: 13 - DNA, 22 bp, synthetic construct
  source 1..22: mol_type = other DNA
ctccaacatcctgaacctcagc

SEQ ID NO: 14 - DNA, 23 bp, synthetic construct
  source 1..23: mol_type = other DNA
cgtcacaccattgctattcttcg

SEQ ID NO: 15 - DNA, 22 bp, synthetic construct
  source 1..22: mol_type = other DNA
gctacagcatgatgcaggacca

SEQ ID NO: 16 - DNA, 22 bp, synthetic construct
  source 1..22: mol_type = other DNA
tctgcgagctggtcatggagtt

SEQ ID NO: 17 - DNA, 22 bp, synthetic construct
  source 1..22: mol_type = other DNA
ttctccagcgacaaggctaagg

SEQ ID NO: 18 - DNA, 22 bp, synthetic construct
  source 1..22: mol_type = other DNA
ctccaacatccaatctgtcccg

SEQ ID NO: 19 - DNA, 23 bp, synthetic construct
  source 1..23: mol_type = other DNA
ccgtaacagacatctttgctgcc

SEQ ID NO: 20 - DNA, 22 bp, synthetic construct
  source 1..22: mol_type = other DNA
gagtgtccttctcatggtggct

SEQ ID NO: 21 - DNA, 20 bp, synthetic construct
  source 1..20: mol_type = other DNA
caagggctaccatgccaact

SEQ ID NO: 22 - DNA, 18 bp, synthetic construct
  source 1..18: mol_type = other DNA
agggccaggaccttgctg

SEQ ID NO: 23 - DNA, 23 bp, synthetic construct
  source 1..23: mol_type = other DNA
cgctacgaagtctgtgacattcc

SEQ ID NO: 24 - DNA, 19 bp, synthetic construct
  source 1..19: mol_type = other DNA
ttccccattgcaggtcatg

SEQ ID NO: 25 - DNA, 23 bp, synthetic construct
  source 1..23: mol_type = other DNA
gatgagcagcttttccagacgac

SEQ ID NO: 26 - DNA, 22 bp, synthetic construct
  source 1..22: mol_type = other DNA
aactggacaccgaacagcagct

SEQ ID NO: 27 - DNA, 22 bp, synthetic construct
  source 1..22: mol_type = other DNA
cggtgaaactctggctagacag

SEQ ID NO: 28 - DNA, 22 bp, synthetic construct
  source 1..22: mol_type = other DNA
gcaaaccgtagatgctcaggga

SEQ ID NO: 29 - DNA, 20 bp, synthetic construct
  source 1..20: mol_type = other DNA
cttccttttcctgggcttcg

SEQ ID NO: 30 - DNA, 21 bp, synthetic construct
  source 1..21: mol_type = other DNA
gaagaccaggaagtgcatcca